(19) United States
(12) Patent Application Publication
(10) Pub. No.: US 2022/0331172 A1
(43) Pub. Date: Oct. 20, 2022

(54) GARMENT FOR TREATMENT OF INCONTINENCE

(71) Applicant: Attn: Grace, Inc., Brookline, MA (US)

(72) Inventors: Alexandra Fennell, Brookline, MA (US); Mia Abbruzzese, Brookline, MA (US); Stephanie L. Howard, Boston, MA (US); Benjamin J. Beck, Boston, MA (US); Michael T. McDuffee, Malden, MA (US); Malory Johnson, Winchester, MA (US); Jennifer Comstock, Boston, MA (US)

(21) Appl. No.: 17/724,876

(22) Filed: Apr. 20, 2022

Related U.S. Application Data
(60) Provisional application No. 63/276,845, filed on Nov. 8, 2021.
(60) Provisional application No. 63/211,125, filed on Jun. 16, 2021.
(60) Provisional application No. 63/177,247, filed on Apr. 20, 2021.

(51) Int. Cl. A61F 13/496; A61F 13/49; A61F 13/494; A61F 13/551

(52) U.S. Cl. CPC A61F 13/496; A61F 13/4906; A61F 13/49426; A61F 13/49011; A61F 13/5512; A61F 2013/8497

(57) ABSTRACT

A garment having an absorbent portion that includes a central absorbent portion having opposing sides, a first gasket portion formed on the central absorbent portion, and a second gasket portion formed on the first gasket portion. In some variations, the garment may include a pair of briefs having an opening for a wearer's torso and leg openings for each of the wearer's legs. The leg openings may include leg cuffs that may include one or more of: light ruching on or inside the leg cuffs, stretch binding or a stretch seam formed on an outer edge of the light ruching, and/or absorbent binding on the leg cuffs.

### CROSS-REFERENCE TO RELATED APPLICATIONS

This application claims priority to and the benefit of U.S. Provisional Patent Application Ser. No. 63/177,247, filed Apr. 20, 2021; U.S. Provisional Patent Application Ser. No. 63/211,125, filed Jun. 16, 2021; and U.S. Provisional Patent Application Ser. No. 63/276,845, filed Nov. 8, 2021, the disclosures of which are incorporated herein by reference in their entireties.

### FIELD OF THE INVENTION

The present invention relates to a garment and, more specifically, to a protective garment for managing absorption of body fluids due to, for example, feminine incontinence.

### BACKGROUND OF THE INVENTION

Historically, individuals who suffer from very heavy bladder leaks (e.g., more than about 105 ml (7 tbsp) of liquid) have often had to resort to wearing a disposable incontinence brief (also referred to as an “adult diaper”). However, there are numerous pain points for wearers of such disposable incontinence briefs, including but not limited to: having to remove their pants and shoes in order to change into a new brief; the fact that briefs are uncomfortable and obvious, requiring the wearer to plan their wardrobe around them; disposing of these briefs is difficult and indiscreet due to their size and bulk; and they both look, feel, and operate as a “diaper” resulting in a demoralizing and undignified experience for the wearer.

In addition to these shortcomings of traditional disposable incontinence briefs, there is substantial waste associated with these products. Specifically, the absorbent and most critical/functional part of the disposable incontinence brief resides in the crotch area, whereas the remainder of the material used to construct these disposable briefs is extraneous and not necessary in order for the brief to perform its intended function (e.g., to capture leaks).

Conventional padded and unpadded garments designed manage to absorption of body fluids resulting from, inter alia, menstruation, incontinence, and so forth include, for example, reusable period underwear, reusable incontinence underwear, disposable incontinence briefs, and disposable incontinence pads. Reusable period underwear, typically, is manufactured from a washable, cotton underwear fabric or a nylon fabric into which an absorbent, felted or carded cotton pad is attached. Reusable period underwear may be configured to provide areas that resist moisture, areas that block the spread of body fluids, and areas that absorb body fluids. These reusable underwear come in an assortment of cuts, including, but not limited to, thong, bikini, cheeky, boy-shorts, hi-waist, hip-huggers, low-rise, mid-rise, French cut, and sleeping shorts. Currently, liquid retention may, however, be limited to a few teaspoon of fluid (e.g., less than about 10 teaspoons).

Reusable incontinence underwear is also configured to provide areas that resist moisture, areas that block the spread of body fluids, and areas that absorb body fluids. These reusable underwear also come in an assortment of cuts, including, but not limited to, thong, bikini, boy-shorts, boy-leg, full brief, hi-waist, hip-huggers, low-rise, mid-rise, French cut, and sleeping shorts. Currently, liquid retention may be limited to a few teaspoon of fluid (e.g., less than about 10 teaspoons) to as much as five to seven ounces.

Disposable incontinence pads must be positioned and, similar to briefs, often have elastic gussets edges. Disadvantageously, there is no absorbency outside of the area of the pad. Wearers also express concern about the smell of discarded pads. As with the incontinence pads, there is no absorbency outside of the area of the pad located within the brief.

As an alternative to incontinence pads, briefs, or underwear—or for concurrent use—is an inter-labial pad. Conventionally, inter-labial pads may be fitted between female labia to absorb menstrual or bladder leaks.

### SUMMARY OF THE INVENTION

In a first aspect, some embodiments of the present invention include a garment having an absorbent portion that includes a central absorbent portion having opposing sides, a first gasket portion formed on the central absorbent portion, and a second gasket portion formed on the first gasket portion. In some applications, the garment further includes a pair of briefs configured to include an opening for a wearer's torso and leg openings for each of the wearer's legs; the opening for the wearer's torso may include a waistband and the leg openings may include leg cuffs. In some variations, the garment may also include one or more of: light ruching on or inside the leg cuffs, stretch binding or a stretch seam formed on an outer edge of the light ruching, and/or absorbent binding on the leg cuffs.

In some implementations, the absorbent portion of the garment is removable and disposable. Moreover, the pair of briefs may include a pocket for removably retaining the absorbent portion. Advantageously, the absorbent portion may also have alignment indicia for aligning the absorbent pad within the pair of briefs. In some variations, the absorbent portion has an adhesive portion for adhering the absorbent portion to the pair of briefs.

In some applications, the second gasket portion comprises a pair of gasket strips disposed longitudinally along the opposing sides.

In a second aspect, embodiments of the present invention include a kit for a disposable protective garment, wherein the kit includes a protective garment having an absorbent portion that includes a central absorbent portion having opposing sides, a first gasket portion formed on the central absorbent portion, and a second gasket portion formed on the first gasket portion; and a disposable bag. In some applications, the garment further includes a pair of reusable briefs configured to have an opening for a wearer's torso and leg openings for each of the wearer's legs and, furthermore, the opening for the wearer's torso may include a waistband and the leg openings may include leg cuffs. In some variations, the garment may also include one or more of: light ruching on or inside the leg cuffs, stretch binding or a stretch seam formed on an outer edge of the light ruching, and/or absorbent binding on the leg cuffs.

In some variations, wherein the disposable bag may include one or more of: an adhesive strip and/or an interlocking strip for adhering a first portion of the disposable bag to a second portion of the disposable bag and/or a selectively removable strip for opening the disposable bag.

In some implementations, the absorbent portion of the garment is removable and disposable (e.g., in the disposable bag). Moreover, the pair of briefs may include a pocket for removably retaining the absorbent portion. Advantageously, the absorbent portion may also have alignment indicia for aligning the absorbent pad within the pair of briefs. In some variations, the absorbent portion has an adhesive portion for adhering the absorbent portion to the pair of briefs.

In a third aspect, embodiments of the present invention relate to an inter-labial pad that is structured and arranged to provide further assurance to the user that leaks will not occur and, more particularly, to use of an inter-labial pad in conjunction with a disposable pad and/or in conjunction with reusable briefs.

In some applications, the second gasket portion comprises a pair of gasket strips disposed longitudinally along the opposing sides.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A shows a protective undergarment, in accordance with some embodiments of the present invention;

FIG. 1B shows the protective undergarment of FIG. 1A having an absorbent material incorporated into the interior portion of the undergarment, in accordance with some embodiments of the present invention;

FIG. 1C shows a protective undergarment with light ruching, in accordance with some embodiments of the present invention;

FIG. 2A shows a protective undergarment with an adhesive disposable pad, in accordance with some embodiments of the present invention;

FIG. 2B shows an adhesive disposable pad, in accordance with some embodiments of the present invention;

FIG. 2C shows a partial plan (top) view of the adhesive disposable pad of FIG. 2B, in accordance with some embodiments of the present invention;

FIG. 3A shows a canyon and gasket-type disposable pad, in accordance with some embodiments of the present invention;

FIG. 3B shows a partial plan (top) view of the canyon and gasket-type disposable pad in FIG. 3A, in accordance with some embodiments of the present invention;

FIG. 3C shows various cross-sectional views of the canyon and gasket-type disposable pad in FIG. 3A, in accordance with some embodiments of the present invention;

FIG. 4A shows an expanding perimeter gasket-type disposable pad in an unexpanded state, in accordance with some embodiments of the present invention;

FIG. 4B shows the expanding perimeter gasket-type disposable pad in FIG. 4A after expanding, in accordance with some embodiments of the present invention;

FIG. 4C shows a cross-section of the unexpanded expanding perimeter gasket-type disposable pad in FIG. 4A, in accordance with some embodiments of the present invention;

FIG. 4D shows a cross-section of the expanded expanding perimeter gasket-type disposable pad in FIG. 4B, in accordance with some embodiments of the present invention;

FIG. 4E shows a partial plan (top) view of the expanded expanding perimeter gasket-type disposable pad in FIG. 4B, in accordance with some embodiments of the present invention;

FIG. 5A shows a perimeter dam-type disposable pad in an inert state, in accordance with some embodiments of the present invention;

FIG. 5B shows the perimeter dam-type disposable pad in FIG. 5A in a damming state, in accordance with some embodiments of the present invention;

FIG. 5C shows a cross-section of the perimeter dam-type disposable pad in FIG. 5B, in accordance with some embodiments of the present invention;

FIG. 5D shows a partial plan (top) view of the perimeter dam-type disposable pad in FIG. 5B, in accordance with some embodiments of the present invention;

FIG. 6A shows a first ridge-and-furrow-type disposable pad, in accordance with some embodiments of the present invention;

FIG. 6B shows a cross-section of the ridge-and-furrow-type disposable pad in FIG. 6A, in accordance with some embodiments of the present invention;

FIG. 6C shows a partial plan (top) view of the ridge-and-furrow-type disposable pad in FIG. 6A, in accordance with some embodiments of the present invention;

FIG. 7A shows a second ridge-and-furrow-type disposable pad, in accordance with some embodiments of the present invention;

FIG. 7B shows a cross-section of the ridge-and-furrow-type disposable pad in FIG. 7A, in accordance with some embodiments of the present invention;

FIG. 7C shows a partial plan (top) view of the ridge-and-furrow-type disposable pad in FIG. 7A, in accordance with some embodiments of the present invention;

FIG. 7D shows a perspective view of the ridge-and-furrow-type disposable pad in FIG. 7A, in accordance with some embodiments of the present invention;

FIG. 8A shows an aquifer-type disposable pad, in accordance with some embodiments of the present invention;

FIG. 8B shows a cross-section of the aquifer-type disposable pad in FIG. 8A, in accordance with some embodiments of the present invention;

FIG. 8C shows a partial plan (top) view of the aquifer-type disposable pad in FIG. 8A, in accordance with some embodiments of the present invention;

FIG. 9 shows a reusable product (e.g., kit) bag having an adhesive seal for sealing a used disposable pad, in accordance with some embodiments of the present invention;

FIG. 10 shows a reusable, foldable wrapper for sealing a used disposable pad, in accordance with some embodiments of the present invention;

FIG. 11 shows a reusable product (e.g., kit) bag with a press-lock seal for sealing a used disposable pad, in accordance with some embodiments of the present invention;

FIG. 12A shows a disposable pad with a plurality of inner and outer gussets, in accordance with some embodiments of the present invention;

FIG. 12B shows a cross-section of the disposable pad shown in FIG. 12A, in accordance with some embodiments of the present invention;

FIG. 12C shows a disposable pad with a plurality of inner and outer gussets in combination with a non-woven mesh layer, in accordance with some embodiments of the present invention;

FIG. 12D shows a cross-section of the disposable pad shown in FIG. 12C, in accordance with some embodiments of the present invention;

FIG. 13 shows a disposable waffle pad, in accordance with some embodiments of the present invention;

FIGS. 14A-14C show a disposable pad having a draw string, in accordance with some embodiments of the present invention; and

FIGS. 15A and 15B show, respectively, first or upper portions and second or lower portions of an inter-labial pad, in accordance with some embodiments of the present invention.

### DETAILED DESCRIPTION

### Protective Undergarment

Protective undergarments typically should be comfortable, unobtrusive, discreet, and clean. Indeed, it is desirable that undergarment material is comfortable and form fitting, ideally, feeling and behaving as do conventional underwear. Inherent in comfort and form fitting is that the protective undergarment is not discernible beneath the wearer's outer garments; hence, the protective undergarment should remain invisible to the eye, ear, and olfactory of others. Functionally, the protective undergarment should be absorbent and, moreover, protect the wearer from leakage.

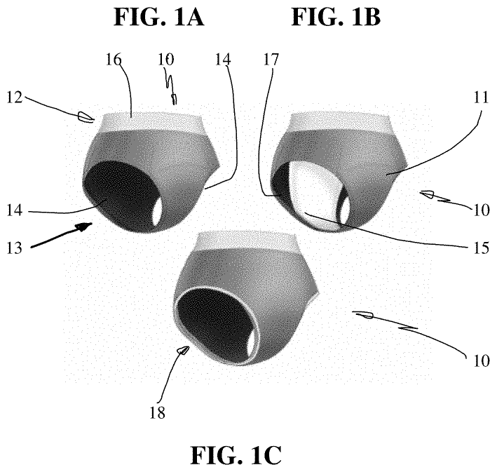

Referring to FIGS. 1A and 1B, an exemplary protective undergarment 10 is shown. The undergarment 10 includes a waist portion 12 and a pair of leg cuffs 14, which provide openings for the wearer's torso and legs, respectively. Variations of reusable protective undergarments 10 may include a high- or mid-waist cut having a waistband 16 that may be folded over another garment, e.g., pantyhose. In some implementations, the protective undergarment 10 includes a front (or crotch) portion 11 and a rear portion 13, as well an interior portion (that contacts the wearer's body) and an exterior portion (that contacts the wearer's outer garments). In some applications, an (e.g., white) absorbent portion 15 structured and arranged to contain and absorb body fluids (e.g., menstrual fluid, blood, urine, and the like) may be integrated into a (e.g., dark) portion 17. The absorbent portion 15 may be configured to cover the interior portion in both the front 11 and the rear portions 13.

For leakage control, as shown in FIG. 1C, in some embodiments, (e.g., light) ruching 18 may be incorporated on or inside of the leg cuffs 14. Although ruching 18 may give the appearance of the leg cuff of a baby's diaper or the corners of a form-fitting bed sheet, stretch binding or stretch seams may be provided on the outer edge of the protective undergarment 10 to provide a cleaner, smoother look and feel. In another application, the leg cuffs 14 may be inward-facing, which arrangement acts as a drip-catch and a gasket on the wearer's skin. Preferably, the inward-facing leg cuff 14 may be sewn into place at a plurality (e.g., two, three, four, or more) of locations to prevent inversion of the inward-facing leg cuff 14.

As an alternative or in addition to ruching 18, an (e.g., super) absorbent material may be incorporated into the edge binding or taping for leakage protection. For example, each leg cuff 14 may include one or more (e.g., two, three, four, or more) miniature ribs made of an absorbent material (e.g., silicone) that creates another line of defense against leakage. In some variations, the miniature ribs are disposed in parallel about the entire periphery or substantially the entire periphery of the opening of the leg cuffs 14. Advantageously, the miniature ribs may be so small that they remain flexible and comfortable until they absorb body fluids.

Although the invention has been described using light ruching 18, that is done for illustrative purposes only. In various embodiments, any amount of ruching 18 can be used. For example, in some applications, a heavy ruching 18 may be more desirable and/or the protective undergarment 10 may include dedicated wings or gusset ruching 18 to protect against leakage when the wearer is in an unusual position.

### Protective Pad

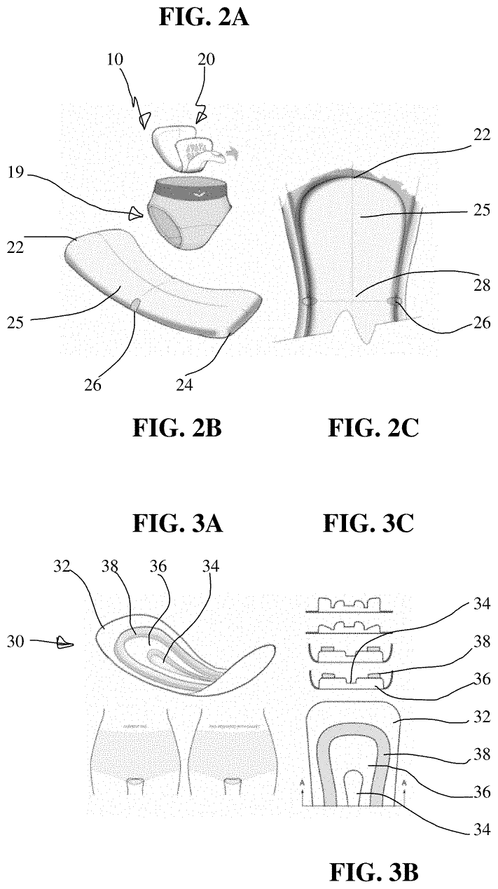

Although, in some embodiments, the absorbent portion 15 of the protective undergarment 10 may be fixedly attached to (e.g., sewn into) the undergarment 10, as shown in FIG. 2A, in another embodiment, a removable and (e.g., variable size and variable purpose) disposable pad 20 may be inserted into the interior portion 19 of the undergarment 10. Advantageously, because the silhouette of each disposable pad 20 may be substantially identical, the wearer can select a protective undergarment 10 having the highest absorption level using the most absorbent pad 20.

Referring to FIGS. 2B and 2C, the disposable pad 20 may include an absorbent (e.g., liquid-permeable, breathable, non-woven cloth) pad material 25 having a distal end 22 and a proximal end 24. Advantageously, the absorbent pad material 25 may include a body fluid collection portion that may be adapted for different leakage amounts. For attaching the pad 20 to the protective undergarment 10, an adhesive portion may be used and/or a pair of flexible tabs 26 may be disposed proximate alignment indicia 28 that may be provided in the interior portion 19 of the undergarment 10. In some variations, the alignment indicia 28 may be a sewn-in or printed graphic showing the wearer where the pad 20 should be aligned in the undergarment 10. Advantageously, the alignment indicia 28 may be color coded to match the various sizes of the disposable pads 20.

For example, in some implementations, an adhesive portion may be placed, affixed, and/or attached on the underside or away side of the disposable pad 20 and covered with a non-adhesive (e.g., double folded) cover having a pull-tab portion. To use, the wearer positions the disposable pad 20 at or near the center point of the protective undergarment 10, holding the disposable pad 20 in place with one hand, while the other hand applies a tensile force to the pull-tab portion, removing the (e.g., non-adhesive) pull-tab portion and exposing the adhesive portion. As an alternative to using an adhesive or flexible tabs 26 to removably attach a pad 20 to an undergarment 10, the undergarment 10 may include a sewn-in pocket in the interior portion 13.

As a further alternative to an adhesive portion, a hook and loop system may be used. For example, the protective garment 10 may be adapted to include one or more strips containing loops and the disposable pad 20 may be configured to contain an equal number of strips containing hooks that, when meshed with the loops on the protective garment 10, position and secure the disposable pad 20 to the protective garment 10. Alternatively, a multiplicity of micro-hooks may be formed on the underside of the disposable pad 20. In use, the micro-hooks are adapted to attach directly to the material of the protective garment 10, which eliminates the need for adhesive strips.

Referring to FIGS. 3A-3C, a first embodiment of a (e.g., molded) disposable pad 30 is show. The disposable pad 30 may be structured and arranged to follow the natural path of the body fluid and the body's natural curvature to optimize absorption. Advantageously, the (e.g., molded) disposable pad 30 creates a sealed gasket within the pad portion 32 itself. In some implementations, the disposable pad 30 includes a (e.g., molded) pad portion 32 having a central (e.g., collection or reservoir) portion 34 about which a (e.g., die-cut) gasket portion 36 made of an absorbent, non-woven material may be fixedly attached to provide a sealable perimeter about the central (e.g., collection or reservoir) portion 34 and a first level of liquid containment. Gasket strips 38 (e.g., die-cut gasket strips) made of an absorbent, non-woven material may be fixedly attached to (e.g., the sides of) and/or incorporated or integrated into the gasket portion 36. Although FIG. 3A shows that the gasket strip 38 completely surrounds the gasket portion 36, this is done for illustrative purposes only. In some variations, the gasket strips 38 are just provided along the longitudinal sides of the gasket portion 36. For a more comfortable transition, the edges of the gasket strips 38 may be skived.

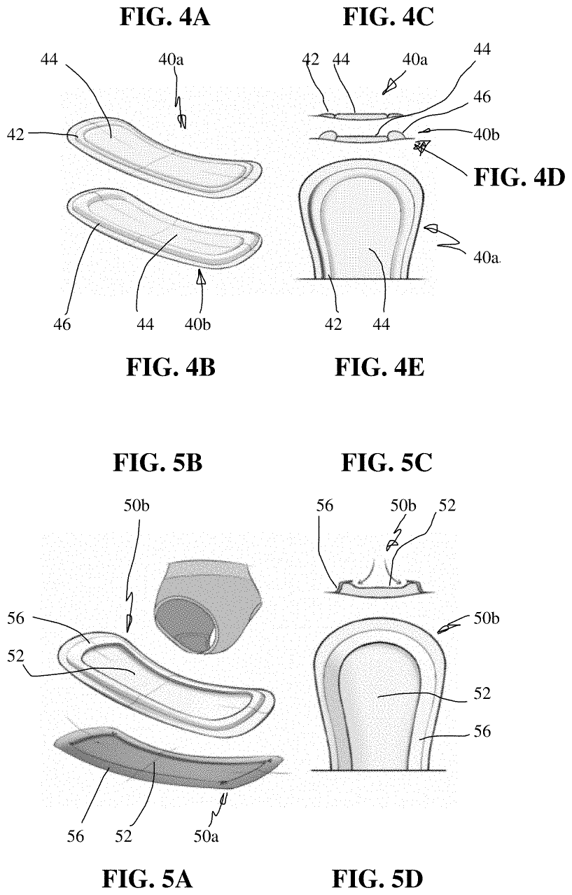

Referring to FIGS. 4A-4E, a second embodiment of a disposable pad 40a, 40b having an (e.g., hydrophilic) expanding perimeter gasket is shown. FIGS. 4A and 4C show the disposable pad 40a with a compressed (e.g., hydrophilic) outer edge 42, while FIGS. 4B and 4D show the disposable pad 40b with an expanded outer edge 46, resulting from absorption of fluids.

In some applications, when in a compressed state, the (e.g., expanding perimeter gasket) disposable pad 40a is structured and arranged to have an absorbent (e.g., central) pad portion 44 about which a compressed outer edge 42 (e.g., a gasket) manufactured of the same or similar absorbent material is disposed. As shown in FIGS. 4B and 4D, after contact with a fluid and in an expanded state, the compressed outer edge 42 absorbs fluid quickly, causing the previously compressed outer edge 42 to expand and swell. Advantageously, the expanding outer edge 46 presses the expanded outer edge 46 against the skin of the wearer. The pressure of the expanded outer edge 46 against the skin of the wearer creates a (e.g., waterproof) seal that is adapted to contain the further flow of body fluid beyond the expanded outer edge 46.

Referring to FIGS. 5A-5D, an embodiment of a perimeter dam-type disposable pad 50a, 50b is shown. In some embodiments, the disposable pad 50a, 50b includes an absorbent (e.g., central) pad portion 52 that is surrounded by a waterproof portion 56. As shown in FIG. 5C, when in contact with a fluid, the absorbent (e.g., central) pad portion 52 absorbs the fluid, causing the (e.g., central) pad portion 52 to raise up as the absorbent (e.g., central) pad portion 52 absorbs fluid and swells. As the (e.g., central) pad portion 52 is raised, the waterproof portion 56 is lifted up and acts as a dam, containing fluid that cannot or is slow to be absorbed within the (e.g., central) pad portion 52. In use, the perimeter dam-type disposable pad 50a, 50b may be inserted in a pocket integrated into the protective undergarment 10.

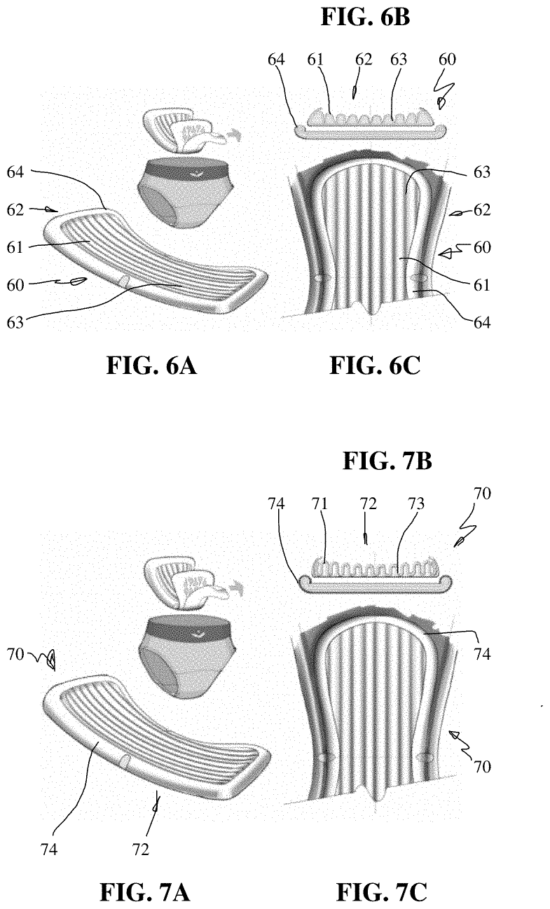

Referring to FIGS. 6A-6C, a first embodiment of a ridge-and-furrow-type disposable pad 60 is shown. In some variations, the ridge-and-furrow-type disposable pad 60 has an absorbent (e.g., central) portion 62 that is surrounded by a peripheral (e.g., absorbent) edge dam side wall 64. The absorbent (e.g., central) portion 62 consists of or consists essentially of a plurality of ridges 61 and furrows 63 running longitudinally from the forward edge of the pad 60 to the rear edge of the pad 60. Advantageously, the furrows 63 promote pooling of fluid away from the surface of the wearer's skin, reducing moisture contact with the skin and promoting breathability.

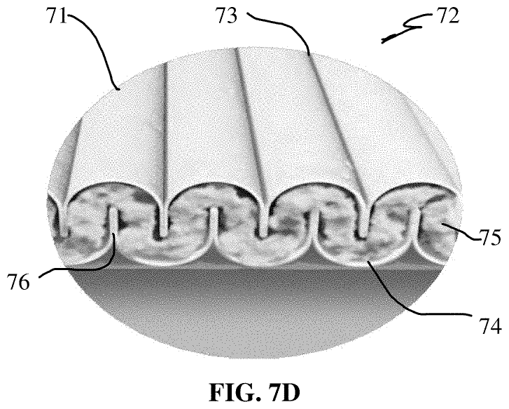

A second embodiment of the ridge-and-furrow-type disposable pad 70 concept is shown in FIGS. 7A-7D. The embodied ridge-and-furrow-type disposable pad 70 also has an absorbent (e.g., central) portion 72 that is surrounded by a peripheral (e.g., absorbent) edge dam side wall 74. The absorbent (e.g., central) portion 72 consists of or consists essentially of a plurality of ridges 71 and furrows 73 running longitudinally from the forward edge of the pad 70 to the rear edge of the pad 70. Advantageously, the ridges 71 and furrows 73 optimize the exposed surface area. Moreover, the furrows 73 promote pooling of fluid away from the surface of the wearer's skin, reducing moisture contact with the skin and promoting breathability.

As shown in FIG. 7B, the arrangement of alternating ridges 71 and furrows 73 provides a distinctive corrugated appearance. Alternatively, as shown in FIG. 7D, the pad 70 may resemble an accordion structure. More particularly, the absorbent (e.g., central) portion 72 may be structured and arranged such that the furrows 73 extend only partially through a first (e.g., upper) side of an absorbent core portion 75. On the second (e.g., under) side of the absorbent core portion 75 may be configured another series of ridges 74 and furrows 76. Preferably, the furrows 76 associated with the second side of the absorbent core portion 75 extend towards the ridges 71 associated with the first side of the absorbent core portion 75 and the furrows 73 associated with the first side of the absorbent core portion 75 extend towards the ridges 74 associated with the second side of the absorbent core portion 75. In use, the ridges 71 and furrows 72 optimize the surface area in the absorbent (e.g., central) portion 72 for temporary pooling before the fluid can be absorbed into the absorbent core portion 75.

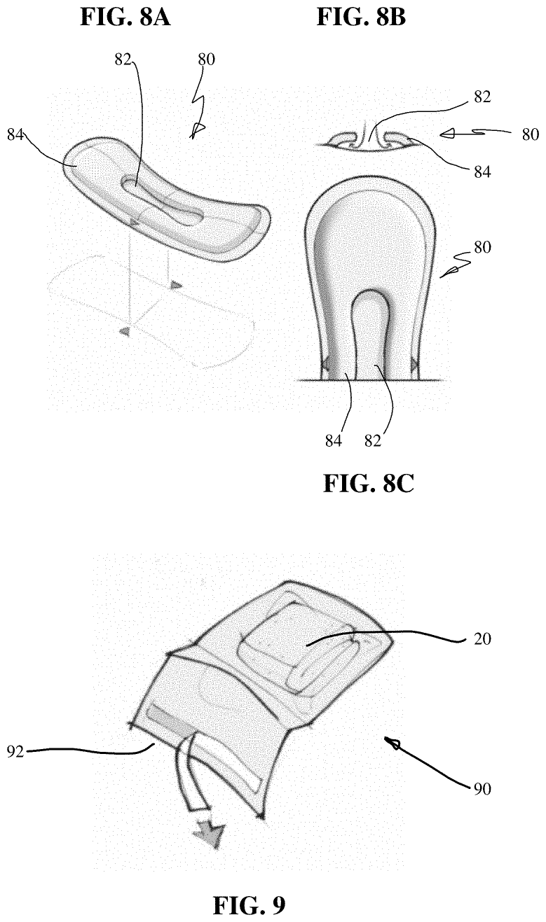

Referring to FIGS. 8A-8C, an aquifer-type disposable pad 80 is shown. In some implementations, the aquifer-type disposable pad 80 includes a central opening 82 and a (e.g., peripheral) fully-sealed pad 84. In operation, body fluid enters the central opening 82 where it pools until it is adsorbed by the surrounding (e.g., peripheral) pad 84. The expanding (e.g., peripheral) pad 84 provides a partial seal as the pad 84 presses against the skin of the wearer.

### Inter-Labial Pad

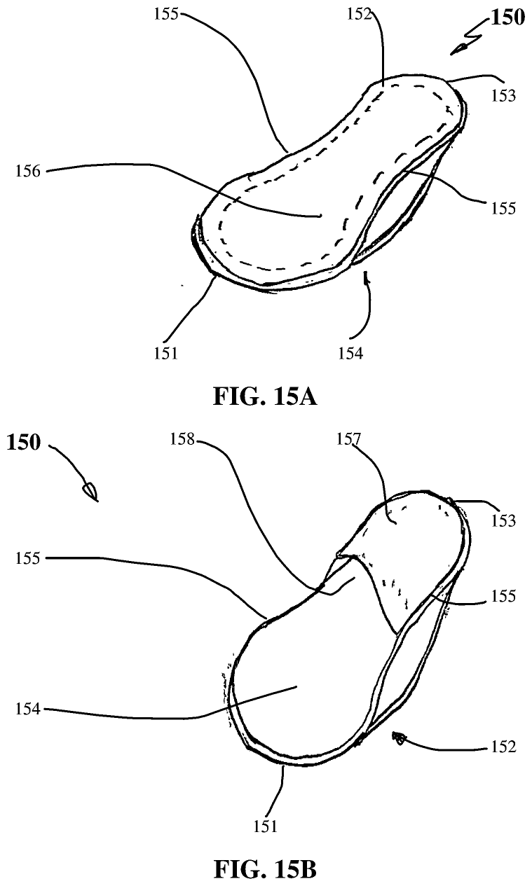

Referring to FIGS. 15A and 15B, an inter-labial pad 150 for providing further assurance to the user to prevent or minimize leaks is shown. In some implementations, the inter-labial pad 150 may be used by itself while in other implementations, the inter-labial pad 150 may be used and integrated with any of the disposable pads discussed hereinabove and/or with reusable briefs discussed hereinabove. Advantageously, use of an inter-labial pad 150 in conjunction with a. disposable pad and/or with reusable briefs may eliminate the need for a wearer to replace the disposable pad and/or reusable briefs as frequently, resulting in less waste and lower costs for the wearer.

In some variations, the inter-labial pad 150 may be configured with a dual structure that may include a first or upper sheet portion 152 and a second or lower sheet portion 154. Advantageously, the first 152 and second sheet portions 154 may be attached, affixed, adhered, bonded, and the like, respectively, to each other at or to a front end 151 of the pad. 150 and a back end 153 of the pad 150. The longitudinal sides 155 of the sheet portions 152, 154 preferably remain unattached or un-bonded to one another. As a result of this arrangement, only the first or upper sheet portion 152 is capable of deforming or being deformed to provide an appropriate convex) shape that may be inserted between a wearer's labia. In some implementations, squeezing the first or upper sheet portion 152 will cause the portion 152 to assume a desired (e.g., convex) shape.

In some embodiments, an absorbent portion 156 may be positioned in a central portion) of the first or upper portion 152. Preferably, the absorbent portion 156 is located beneath an outer layer through which liquid may pass freely.

In some implementations, a finger portion 157 may be attached, affixed, bonded, and the like at the back end 153 of the pad 150 and around some portion of the longitudinal sides 155. Advantageously, the finger portion 157 is structured and arranged to provide an opening 158 and a plenum space between the finger portion 157 and the second or lower portion 154, such that the wearer may insert their finger into the plenum space to insert or remove the pad 150.

### Protective Undergarment Kit

Having described a protective undergarment, a kit and method of disposing of the protective garment will now be described. Advantageously, referring to FIG. 9, the packaging 90 in which the protective undergarment 10 or, alternatively, the disposable pad 20, is purchased may be saved and used to wrap or contain the used protective undergarment 10 and/or disposable pad 20 for disposal. In some implementations, the packaging 90 may simply be wrapped around the used disposable pad 20 or, as shown in FIG. 9, the used disposable pad 20 may be placed inside the packaging 90. An adhesive portion 92 (e.g., an adhesive strip) may be incorporated into the packaging 90 (e.g., at the top of the packaging 90) to more securely seal the used protective undergarment 10 and/or disposable pad 20 within the packaging 90.

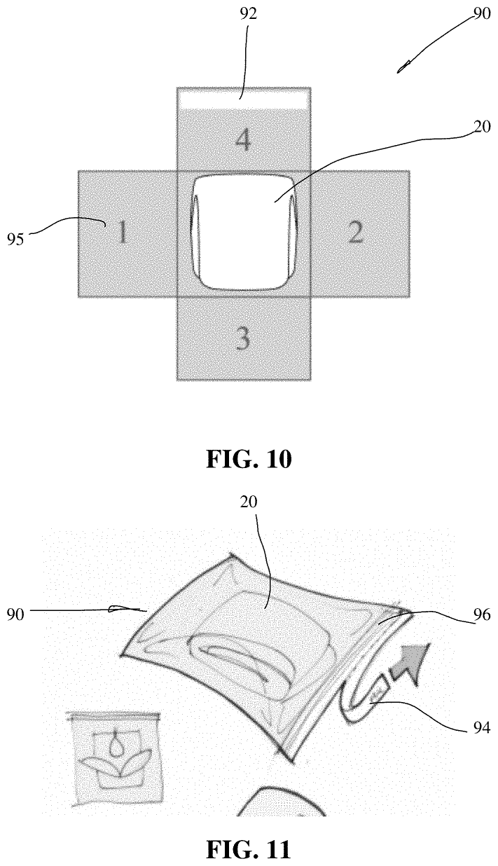

In another embodiment, as shown in FIG. 10, the packaging 90 of a replacement protective undergarment 10 and/or disposable pad 20 may be taken apart and used to dispose of the used protective undergarment 10 and/or pad 20. For example, the packaging 90 may, when broken down, include a plurality of sidewalls 95 that may be folded over the used protective undergarment 10 and/or disposable pad 20 and each other. Preferably, one of the sidewalls 95 (e.g., sidewall number 4) may include an adhesive portion 92 (e.g., strip) to more securely seal the packaging 90.

Referring to FIG. 11, in yet another embodiment, when purchased, the protective undergarment 10 or, alternatively, the disposable pad 20 may come sealed (e.g., air-proof, water-proof, or the like) within packaging 90 that includes a selectively removable tear-off strip 94. Once the tear-off strip 94 has been removed from the packaging 90, the wearer may remove the new protective undergarment 10 or the new disposable pad 20 from inside the packaging 90. The used protective undergarment 10 or disposable pad 20 may then be inserted into the packaging 90. In some variations, a (e.g., interlocking) sealing device 96 disposed at or near the opening of the packaging 90 may be used to seal the packaging 90 once a used protective undergarment 10 or disposable pad 20 has been placed within the packaging 90.

### Additional Protective Undergarment Features

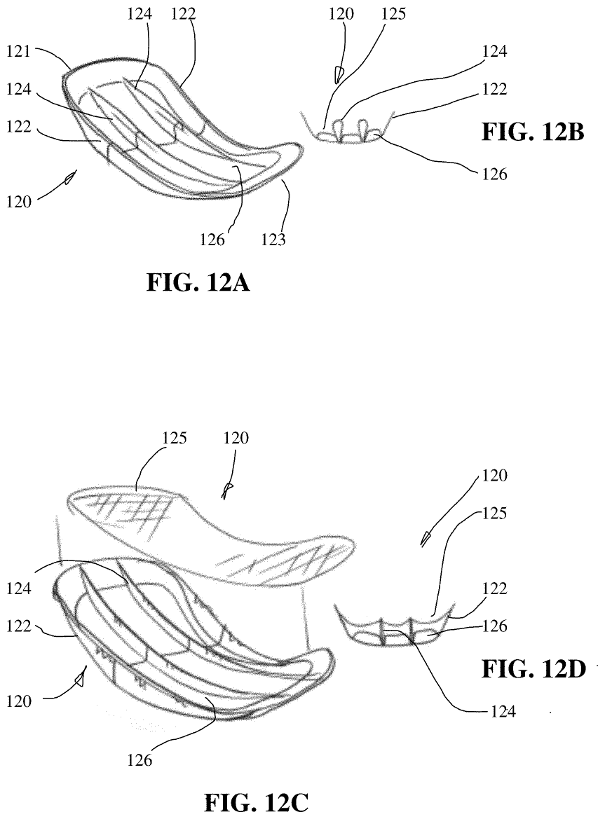

Referring to FIGS. 12A-12D, embodiments of a disposable pad 120 having a plurality of gussets 122, 124 disposed on or about a padded portion 126 are shown. For example, in some implementations, the disposable pads 120 include a pair of side or outer gussets 122 attached to the padded portion 126 and extending and tapering from the back portion 121 of the padded portion 126 to the front portion 123 of the padded portion 126. A plurality of (e.g., two, three, four, or more) inner gussets 124 may be formed between and substantially parallel to the outer gussets 122. The plurality of inner gussets 124 allows for more channeling protection and containment within the padded portion 126 without additional bulk. As shown in FIG. 12B, the inner gussets 124 may be configured to include a wider cross-section and/or may be filled with an absorbent material for greater comfort to the wearer. As an alternative to wider inner gussets 124, as shown in FIGS. 12C and 12D, the inner gussets 124 may be structured and arranged to support a layer of (e.g., non-woven) mesh material 125 that provides greater comfort to the wearer.

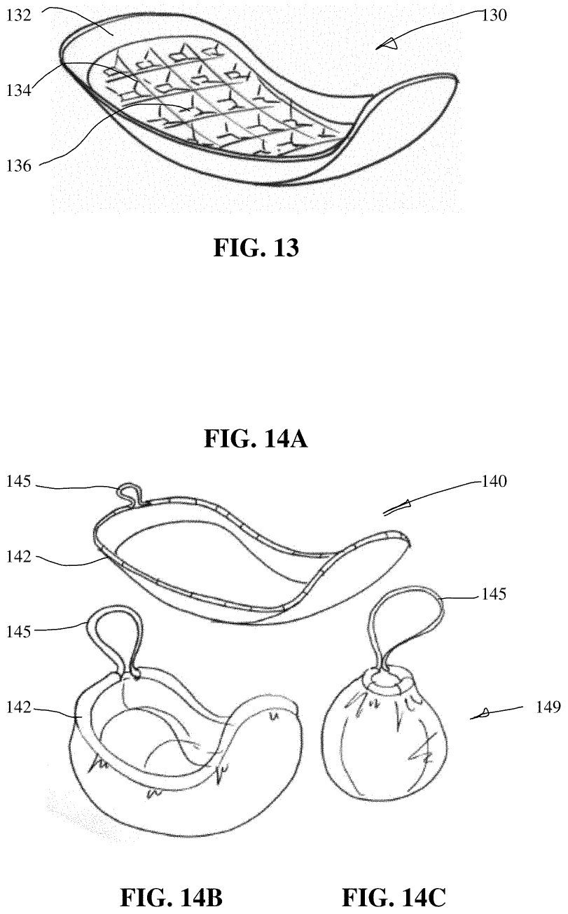

Referring to FIG. 13, a disposable waffle pad 130 is shown. The pad 130 may include a (e.g., absorbent) padded portion 132 onto which grid of ridges 134 have been formed to produce a plurality of (e.g., inverted rectangular pyramidic) cells 136 that are configured to contain (e.g., pool) any leakage until the body fluid is absorbed into the (e.g., absorbent) padded portion 132. Although the cells 136 shown in FIG. 13 are inverted rectangular pyramidic in shape, this is done for the purpose of illustration rather than limitation. Those of ordinary skill in the art can appreciate that the cells 136 can take on any practical shape that can be formed easily and economically in the padded portion 132. For example, additional variations of cells 136 can include frustro-conical cells, triangular pyramidic cells, hemispherical cells, and so forth.

To minimize the visibility of disposable pad 20, at the edge of the pad 20 or at transition locations, a gradation of seams (e.g., similar to a down skiing jacket) may be formed in the disposable pad 20.

As shown in FIG. 14A, the disposable pad 140 may be structured and arranged to include a drawstring 145 disposed about the peripheral edge 142 of the pad 140. Once the pad 140 has been used, the wearer can pull the draw string 145 (FIG. 14B), causing the pad to ball up 149 (FIG. 14C) for disposal.

Having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A garment comprising:
an absorbent portion comprising:
a central absorbent portion having opposing sides;
a first gasket portion formed on the central absorbent portion; and
a second gasket portion formed on the first gasket portion.

2. The garment of claim 1, wherein the absorbent portion is removable and disposable.

3. The garment of claim 1, wherein the second gasket portion comprises a pair of gasket strips disposed longitudinally along the opposing sides.

4. The garment of claim 1 further comprising:
a pair of briefs having an opening for a wearer's torso and leg openings for each of the wearer's legs, the opening for the wearer's torso comprising a waistband and the leg openings comprising leg cuffs.

5. The garment of claim 4 further comprising light ruching on or inside the leg cuffs.

6. The garment of claim 5 further comprising stretch binding or a stretch seam formed on an outer edge of the light ruching.

7. The garment of claim 4 further comprising absorbent binding on the leg cuffs.

8. The garment of claim 4, wherein the absorbent portion comprises alignment indicia for aligning the absorbent pad within the pair of briefs.

9. The garment of claim 4, wherein the absorbent portion comprises an adhesive portion for adhering the absorbent portion to the pair of briefs.

10. The garment of claim 4, wherein the opening for the wearer's torso comprises a waistband.

11. The garment of claim 4, wherein the pair of briefs comprises a pocket for removably retaining the absorbent portion.

12. A kit for a disposable protective garment, the kit comprising:
a protective garment comprising:
an absorbent portion comprising:
a central absorbent portion having opposing sides;
a first gasket portion formed on the central absorbent portion; and
a second gasket portion formed on the first gasket portion; and
a disposable bag.

13. The kit of claim 12, wherein the absorbent portion is removable and disposable.

14. The kit of claim 12, wherein the second gasket portion comprises a pair of gasket strips disposed longitudinally along the opposing sides.

15. The kit of claim 12, wherein the garment further comprises:
a pair of briefs having an opening for a wearer's torso and leg openings for each of the wearer's legs, the leg openings comprising leg cuffs.

16. The kit of claim 15, wherein the pair of briefs comprises a pocket for removably retaining the absorbent portion.

17. The kit of claim 15, wherein the protective garment further comprises light ruching on or inside the leg cuffs.

18. The kit of claim 17 further comprising stretch binding or a stretch seam formed on an outer edge of the light ruching.

19. The kit of claim 15, wherein the protective garment further comprises absorbent binding on the leg cuffs.

20. The kit of claim 15, wherein the absorbent portion comprises alignment indicia for aligning the absorbent pad within the pair of briefs.

21. The kit of claim 15, wherein the absorbent portion comprises an adhesive portion for adhering the absorbent portion to the pair of briefs.

22. The kit of claim 12, wherein the disposable bag comprises an adhesive strip for adhering a first portion of the disposable bag to a second portion of the disposable bag.

23. The kit of claim 12, wherein the disposable bag comprises a selectively removable strip for opening the disposable bag.

24. The kit of claim 12, wherein the disposable bag comprises an interlocking strip for adhering a first portion of the disposable bag to a second portion of the disposable bag.
